(19) United States
(12) Patent Application Publication
(10) Pub. No.: US 2018/0015240 A1
(43) Pub. Date: Jan. 18, 2018

(54) HANDHELD NEBULIZER

(71) Applicant: Outstanding Healthcare Company Limited, Hong Kong (CN)

(72) Inventors: Ze Guang Zheng, Hong Kong (CN); Kwok Fu FU, Hong Kong (CN); Jie Bing XU, Hong Kong (CN)

(21) Appl. No.: 15/432,135

(22) Filed: Feb. 14, 2017

(30) Foreign Application Priority Data
Jul. 18, 2016 (CN) 201610564979.4

(51) Int. Cl. A61M 15/00; A61M 11/00; A61M 11/04

(52) U.S. Cl. CPC A61M 15/0063; A61M 15/0091; A61M 11/042; A61M 11/001; A61M 2205/502; A61M 2205/6063

(57) ABSTRACT

A handheld nebulizer includes a body having a control assembly and an actuation assembly. The control assembly is in electrical communication with the actuation assembly for delivering nebulized particles of a compound to a subject. A nebulizing chamber accommodates a removable cartridge for conducting nebulization. The removable cartridge holds a mixture of a compound to be nebulized into nebulized particles. A nebulizing element is affixed or detachably mounted to the removable cartridge, and the removable cartridge is in electrical communication with the control assembly. A removable cartridge for a handheld nebulizer is also provided.

### TECHNICAL FIELD

The present invention relates to a handheld nebulizer, in particular but not exclusively to a handheld nebulizer having a removable cartridge for administrating nebulized particles to a subject.

### BACKGROUND

Handheld nebulizer is commonly used to turn a liquid medicament or mixture into nebulized particles such as in the form of vapors. A user can then breathe in the nebulized particles. Such a drug delivery approach is generally applied for treating respiratory diseases or symptoms in a subject in need thereof such as a subject suffering from asthma, cough, fever, sore throat, nasal inflammation etc.

However, the existing handheld nebulizers are generally susceptible to contamination by dirt, bacteria and/or unwanted particles and such a contamination may be associated with an increase in health risks. This is because the existing nebulizers generally require refilling of the liquid medicament to a liquid storage chamber through, for example, syringe injection or replacement of a liquid filing container, without thoroughly cleaning the liquid storage chamber. The nebulizing elements affixed in these nebulizers are repeatedly used and these nebulizing elements cannot be thoroughly cleaned, sterilized or replaced. Further, the existing approaches for refilling, cleaning or replacement are limited and sometime require complicated procedures that a user may not be able to operate.

Moreover, the current handheld nebulizers generate a cool mist from liquid medicament in nebulization for a user to inhale. However, since the temperature of the cool mist is lower than body temperature, the user usually feels uncomfortable when administering the mist. The cool mist may also quickly condense in unwanted places leading to contamination and/or further trigger irritation to the respiratory tract and thus the desired therapeutic effect cannot be attained, or may not be efficiently attained.

Accordingly, there remains a need for an improved handheld nebulizer in which the aforesaid shortcomings are mitigated or at least to provide a useful alternative to the trade and public.

### SUMMARY OF THE INVENTION

The present invention provides a handheld nebulizer including a body having a control assembly and an actuation assembly. The control assembly is in electrical communication with the actuation assembly for delivering nebulized particles of a compound to a subject. A nebulizing chamber accommodates a removable cartridge for conducting nebulization. The removable cartridge holds a mixture of a compound to be nebulized. A nebulizing element is affixed or detachably mounted to the removable cartridge, and the removable cartridge is in electrical communication with the control assembly.

The present invention also pertains to a removable cartridge for a handheld nebulizer, including a nebulizing element.

Without intending to be limited by theory, it is believed that the handheld nebulizer of the present invention may address one or more of the problems discussed above. In particular, the handheld nebulizer of the present invention is easy to operate and can substantially prevent/minimize contamination by unwanted dirt, bacteria and/or particles on the nebulizing element thereby minimizing health risks caused by such a contamination. The heating element, if present, can further help to reduce undesired irritations caused by the inhalation of cool air or nebulized particles.

It has been found that the handheld nebulizer and the removable cartridge of the present invention are exceptionally suitable for large scale production. The provision of the identification element allows a single handheld nebulizer to correctly and automatically identify various cartridges holding different compounds for carrying out the respective nebulization. This in turn reduces patient error and the chance of improper administration and/or dosages of medicines. In addition, the cartridge used herein may be quick, easy, and inexpensive to produce and may be disposable, which further reduces the chance of contamination.

### BRIEF DESCRIPTION OF THE DRAWINGS

Embodiments of the present invention will now be described, by way of example, with reference to the accompanying drawings. In the drawings, identical components/elements are denoted with the identical numerical reference, wherein like numerical references refers to the similar elements. In the present application, the numerical references of similar or identical components in different embodiments (such as 100, 200) may have a number difference of 100 so as to indicate their similarity

FIG. 1 illustrates an embodiment of a handheld nebulizer according to the present invention.

FIG. 2 shows the handheld nebulizer of FIG. 1 with the cover and a part of the housing of the nebulizing chamber removed.

FIG. 3A to FIG. 3G show different views of an embodiment of a removable cartridge according to the present invention; wherein FIG. 3A shows the perspective view, FIG. 3B shows the front view, FIG. 3C shows the rear view, FIG. 3D shows the top view, FIG. 3E shows the bottom view, FIG. 3F shows the right side view and FIG. 3G shows the left side view.

FIG. 4 illustrates an embodiment of a handheld nebulizer according to the present invention.

FIG. 5 illustrates an embodiment of a handheld nebulizer according to the present invention.

FIG. 6 is a diagram showing the actuation assembly applied in the handheld nebulizer of FIG. 5 having an infrared sensor and a movable block.

The drawings herein are for illustrative purposes only and may not be drawn to scale.

### DETAILED DESCRIPTION OF THE INVENTION

The present invention provides a handheld nebulizer including a body having a control assembly and an actuation assembly. The control assembly is in electrical communication with the actuation assembly for delivering nebulized particles of a compound to a subject. A nebulizing chamber accommodates a removable cartridge for conducting nebulization. The removable cartridge holds a mixture of a compound to be nebulized, wherein a nebulizing element is affixed or detachably mounted to the removable cartridge, and wherein the removable cartridge is in electrical communication with the control assembly.

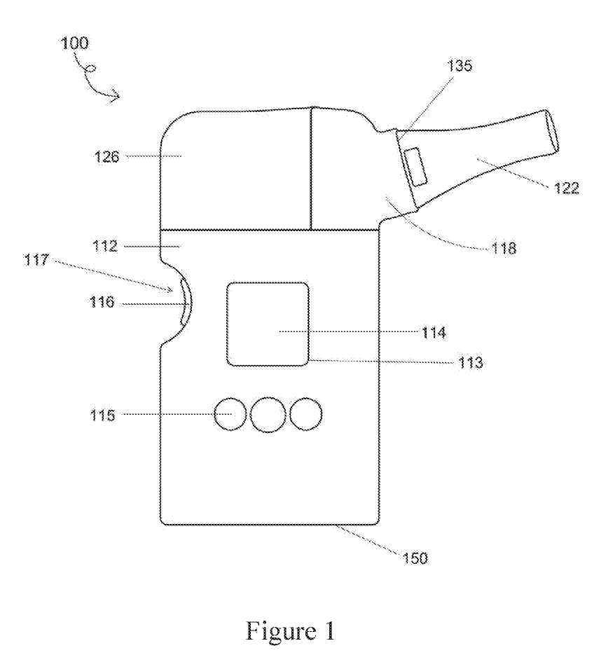

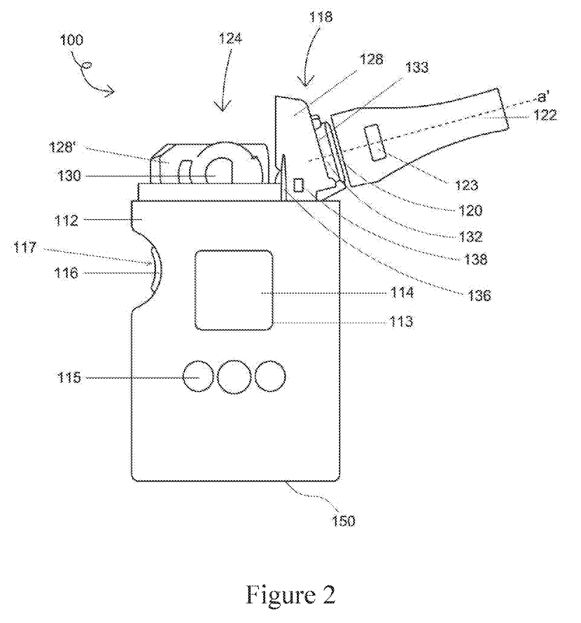

FIGS. 1 and 2 show a handheld nebulizer according to an embodiment of the present invention. The handheld nebulizer 100 includes a body 112 which is arranged with a control assembly 113 having a display screen 114 and input buttons 115 on one surface of the body 112 for operating the handheld nebulizer 100. The body 112 is also provided with an actuation assembly 117 in connection with the control assembly 113, the actuation assembly 117 includes an actuation element such as an actuation button 116 on a side surface of the body 112 to enable a user to actuate the nebulization for use.

A nebulizing chamber 118 accommodating a removable cartridge 128 is provided above the body 112. The nebulizing chamber 118 has an outlet 120 which is in fluid communication with a guiding nozzle 122 coupled to the nebulizing chamber 118. The guiding nozzle 122 extends outwardly from the nebulizing chamber 118 and has two opposite holes 123 on its wall for guiding the flow of nebulized particles. The arrangement of the holes 123 on the guiding nozzle 122 enhances the flow of the nebulized particles when the user inhales, i.e. the suction force generated by the user draws the surrounding air to enter from the holes 123 and provides an additional force for moving the nebulizer particles towards the user in a more efficient manner. Preferably, the direction of the flow of the nebulized particles leaving the outlet 120 is in parallel with the extension direction of the guiding nozzle 122, i.e. along the axis a′.

Next to the nebulizing chamber 118 is a storage chamber 124 provided with a cover 126. In FIGS. 1 and 2, the storage chamber 124 is optionally-arranged to accommodate an additional removable cartridge 128′ for spare use. The additional removable cartridge allows the user to quickly replace a depleted cartridge with a new and optionally prefilled one. Such an arrangement is particularly advantageous in that it enables the user to easily replace with a new one when he/she is travelling and cannot access to the stock at home. Such an arrangement also avoids complicated replacement steps and reduces the potential for contamination.

With reference to FIG. 2, a removable cartridge 128 is fitted into the nebulizing chamber 118 for nebulization. The removable cartridge 128 is typically prefilled with a mixture of a compound to be nebulized through an opening 130 and is provided with a nebulizing element 132 to cover the opening 130. In this embodiment, the nebulizing element 132 is arranged with a seal ring 133 and is detachably mounted on the removable cartridge 128. Before being fitted in the nebulizing chamber 118, the removable cartridge 128 is optionally additionally protected by a thin membrane (not shown) to prevent the nebulizing element from contacting with any dirt or contamination before use. Alternatively, the nebulizing element 132 may be permanently affixed to the removable cartridge 128 by an adhesive, a fastener, etc., for example, the nebulizing element 132 may be attached to the removable cartridge 128 or fastened by a clip and/or screws. In this embodiment, the nebulizing element 132 includes an ultrasonic atomizer for nebulizing the mixture. Other alternative atomizers known in the technical field may be applied.

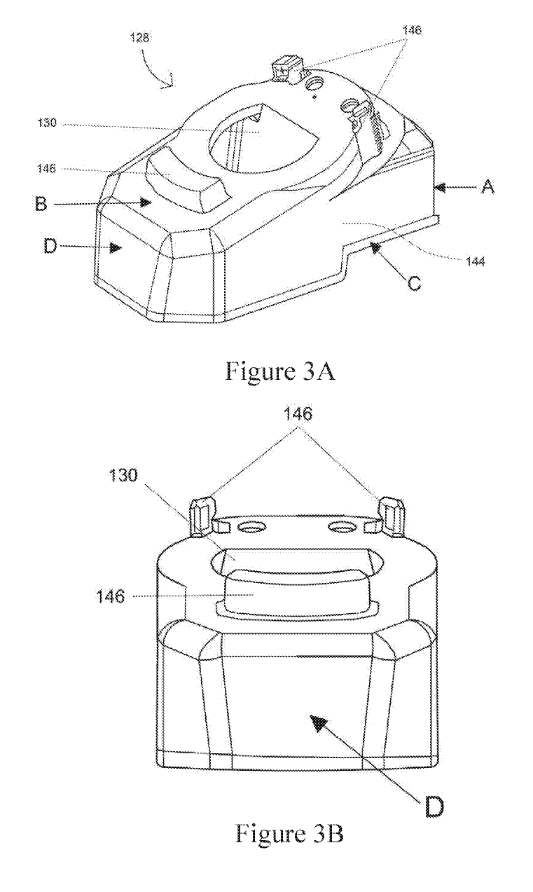

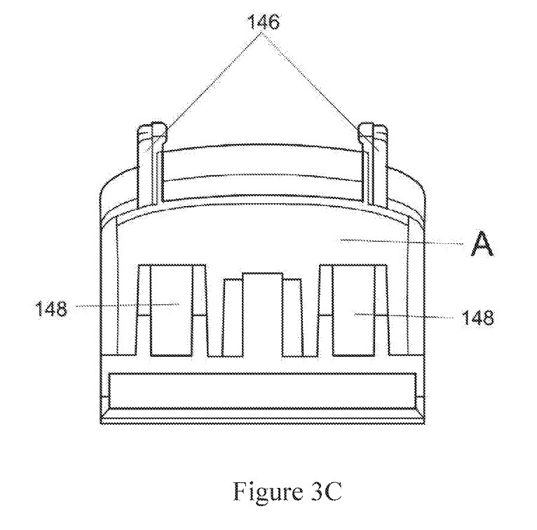

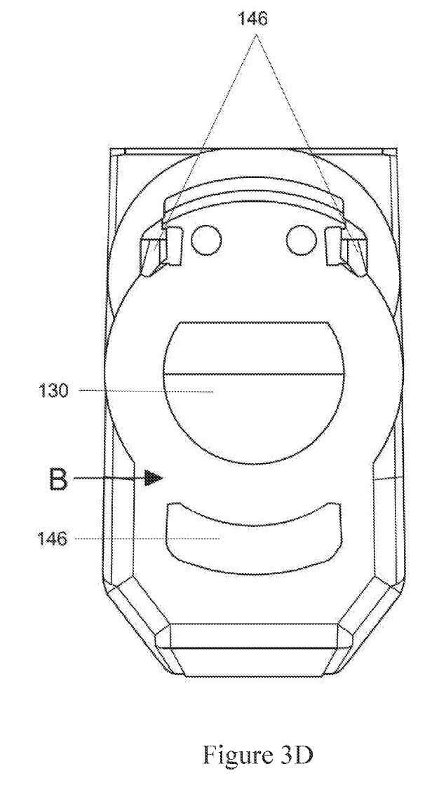

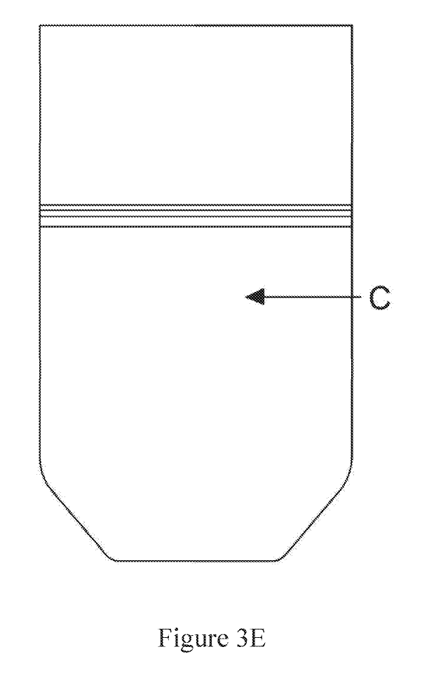

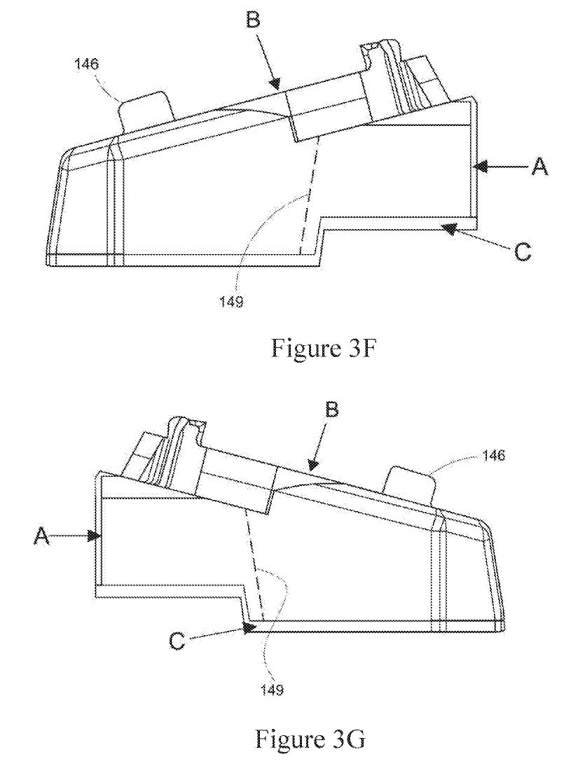

When the removable cartridge 128 is fitted into the nebulizing chamber 118, it is in electrical communication with the control assembly 113. In particular, the removable cartridge 128 may be provided with one or more electronic contacts (see FIG. 3 at 148) to interact with the control assembly 113 and thus when the nebulizer is actuated, the nebulizing element 132 is triggered to nebulize the mixture so as to produce nebulized particles. Alternatively, the removable cartridge 128 may be provided with one or more physical contacts and/or electrical chips to interact with the control assembly 113.

Preferably, in an embodiment herein, the nebulized particles have an average size of about or less than 5 μm, for example from about 1 μm to about 5 μm; or less than about 1 μm; or about 1 μm; or about 2 μm; or about 3 μm; or about 4 μm; or about 5 μm; or from about 1.5 μm to about 4.5 μm; or from about 2.5 μm to about 4 μm; or any combination of the above values. The skilled person is aware of suitable methods for determining the average size of particles, or called the average mass median aerodynamic diameter of particle. For example, the size of the particle is determined by the method described in the European Standard EN 13544-1:2007+A1:2009(E) entitled: Respiratory therapy equipment—Part 1: Nebulizing systems and their components. In particular, the particle sizing method is described in Annex BB and CC.

The nebulizing chamber 118 further includes an output seal ring 135 which is adapted to couple with the removable cartridge 128. The output seal ring 135 abuts the nebulizing element 132 and holds the removable cartridge 128 in place and/or avoid leakage of any mixture or nebulized particles in between the removable cartridge 128 and the nebulizing chamber 118. The seal ring 135 also helps to reduce the chance of contamination and may prevent back flow of the nebulized particles into the removable cartridge 128 or nebulizing chamber 118.

When using the handheld nebulizer 100, a user can quickly and easily fit a removable cartridge 128 into the nebulizing chamber 118 and switch on the handheld nebulizer. The display screen 114 then shows parameters of the nebulization and/or working modes of nebulization. Parameters of the nebulization may include, for example, the length of time of the nebulization process, the time interval, temperature, the speed of the nebulization, etc. The working modes of nebulization typically refer to preset nebulization modes/patterns. The user can select a suitable mode from various available choices for conducting the nebulization. Accordingly, the user may select the predetermined setting or adjust parameters of the nebulization through the input buttons 115 according to the user's actual requirements. When a desired working mode is selected, the user holds the handheld nebulizer 100, typically directs the guiding nozzle 122 towards his/her mouth and/or nose, and actuates the nebulization by pressing the actuation button 116. The nebulizing element of the removable cartridge 128 is then triggered to nebulize the mixture and produce nebulized particles. The nebulized particles travel along the guiding nozzle 122 and are inhaled by the user. The nebulization may continue until the actuation button 116 is released. Alternatively, the user may select a working mode that follows a user's breathing pattern. In such a case, the nebulization may follow the user's breathing pattern.

In an embodiment of the present invention, the handheld nebulizer 100 further includes a heating element 136. The heating element 136 may be arranged in the nebulizing chamber 118 and/or attached on/in the removable cartridge 128 for heating the mixture in the removable cartridge 128 before the nebulization. Without intending to be limited by theory, it is believed that a heating element is particularly advantageous to provide the nebulized particles with a suitable average temperature for the user to inhale. The user can then inhale warm nebulized particles to relieve any symptoms or illness.

Preferably, the nebulized particles delivered after the heating and nebulization have an average temperature of from about 20° C. to about 40° C., from about 23° C. to about 37° C., or from about 25° C. to about 35° C. In one embodiment, the average temperature is from about 25° C. to about 35° C., 25° C., 26° C., 27° C., 28° C., 29° C., 30° C., 31° C., 32° C., 33° C., 34° C. or 35° C. The skilled person is aware of suitable means to determine the average temperature of the nebulized particles for example using a negative temperature coefficient type sensor, and would appreciate that different compounds may be nebulized and delivered at different temperature according to their physicochemical properties. Average temperature may be obtained by repeating the measurement for at least three times. The skilled person appreciates that various heating elements may be applied to the present invention to heat the mixture. For example, the heating element may be selected from the group consisting of a heating coil, a heating plate, a copper tube, and a combination thereof.

In a further embodiment of the present invention, the handheld nebulizer 100 further includes an identification element 138 on the removable cartridge 128. The identification element 138 can be recognized by the control assembly 113 to determine the identity of the removable cartridge 128. The identity consists of one or more pieces of information, for example, components of the mixture loaded in the removable cartridge 128, i.e. types of compound, carrier (e.g. solvent) and the like, instructions for use, and/or the application of the mixture.

In FIG. 1, the identification element 138 is a RFID tag, but one skilled in the art understands that the identification element may alternatively be a designed structure on the removable cartridge integrated in to the cartridge, a separate element attached to the cartridge, etc. The identification element may be selected from the group consisting of a RFID tag, an electronic chip card, a printed label, a designed integrated structure and a combination thereof. In the embodiment of FIGS. 1-2, the control assembly 113 is configured to recognize the identity of the removable cartridge 128 and adjusts preset parameters of the handheld nebulizer 100 and/or selects a respective preset mode according to the identity of the removable cartridge 128. The control assembly 113 may detect the identity of the removable cartridge 128 directly or indirectly.

It is appreciated that in an embodiment of the present invention, the control assembly may include a display, a timer, a sensor, an input button, a circuit board, a database or a combination thereof; and the actuation element of the actuation assembly may include a switch, button, a sensor or a combination thereof, or any component that can react with the user to deliver an actuation signal. Other possible obvious variations or modifications may also be applied to the present invention without departing from the spirit of the present invention.

It is also appreciated that the mixture of a compound to be nebulized may include a medicament, an essential oil, and/or an excipient. The mixture may be a solution, a dispersion, a suspension, or a colloid; or a solution. The mixture may also be a pure compound.

The present invention also pertains to a removable cartridge for a handheld nebulizer. FIG. 3 shows different views of an embodiment of a removable cartridge of the present invention that may be adapted to the handheld nebulizer 100 as described above. As shown in FIG. 3, the removable cartridge 128 includes an opening 130 and a cavity 144 for holding a mixture to be nebulized. The protrusions 146 as shown in the figure are configured to receive a nebulizing element 132 between the opening 130 and the protrusions 146.

The nebulizing element is arranged with a seal ring 133 to connect and abut the protrusions 146. When the removable cartridge 128 is loaded with the mixture, the nebulizing element 132 is then affixed to or detachably mounted to the removable cartridge 128 to cover the opening 130.

The removable cartridge 128 is shaped to be fitted in a respective handheld nebulizer. In this embodiment, when the removable cartridge 128 is fitted into a nebulizing chamber 118 having a similar design as shown in FIGS. 1 and 2, a surface A of the cartridge faces towards the body, i.e. downwards, and a surface B faces towards the outlet of the nebulizing chamber. The electrical contacts 148 arranged on the surface A are then in contact with the control assembly of the handheld nebulizer. In use, the control assembly (e.g. as shown as 113 in FIG. 1) may send an actuation signal to the nebulizing element 132 through the electrical contacts 148 which is also in electrical connection with the nebulizing element 132. The step-like surface C allows the mixture to flow down towards surface A and at the same time keeps the cartridge firmly in the nebulizing chamber. Preferably, the removable cartridge 128 includes an inner wall 149 (as indicated by a dotted line in FIGS. 3F and 3G). The inner wall 149 and the surface B form an acute angle of less than 90° C. As such, the contact between the mixture and the nebulizing element 132 is maximized for efficient nebulization and to reduce waste. To sum up, the provision of the step-like surface C, surface B and inner wall 149 improves the efficiency of the nebulization. It is appreciate that other shapes of the cartridge can also be applicable.

In an embodiment herein a heating element and/or an identification element may be applied in the cartridge of the invention to achieve the desired improved effects.

It is found that, by way of the arrangements and structures of the handheld nebulizer and removable cartridge as discussed above in particular the application of a removable cartridge, the handheld nebulizer substantially reduces contamination of unwanted dirt or particles on the nebulizing element thereby minimizing health risks caused by such a contamination. The handheld nebulizer is also easy to operate, especially when the mixture to be nebulized is completely depleted, a new cartridge is readily available to replace the depleted one. Also, complicated and potentially dangerous procedures such as using a syringe to refill the mixture are thus avoided.

Furthermore, and without intending to be limited by theory, it is believed that a handheld nebulizer of the present invention having the heating element can reduce undesirable irritation caused by the inhalation of cool air or nebulized particles, especially in frail or elderly users. It is believed that warmer nebulized particles and/or air is less likely to cause a constriction of the airway of the user during inhalation, and is therefore more easily drawn into the body, for example, the mouth, the nasal passages, the esophagus, the lungs, etc. The inventors believe that this is especially true for those who are used to breathing humid and/or warm air, such as those people who are living in the tropics or from such locations. The user can thus more easily and comfortably inhale relatively warm nebulized particles to relieve any pain or illness. The control assembly which is in communication with the heating element may adjust the heating temperature according to the surrounding temperature as detected by a temperature sensor 150 (see FIGS. 1-2). In an embodiment herein, a temperature sensor 150 is provided in the handheld nebulizer. Therefore, the parameters can be easily adjusted according to the change of weather and it does not require many efforts from the user to do so.

Without intending to be limited by the theory it is believed that when the nebulized particles are inhaled by the user with a suitable average temperature in particular with an average temperature close to the body temperature or close to internal body temperature, it is believed that the absorption efficiency of the compound/medicament or the therapeutic efficacy is enhanced.

In addition, a removable cartridge of the present invention having an identification element enables the handheld nebulizer to recognize, for example, what compound is present in the cartridge and which working mode is suitable for said compound. The control assembly may then automatically adjust to a predetermined optimized mode for nebulizing said compound. The provision of identification element is advantageous in that various types of removable cartridge can be manufactured and each of them is assigned with a specific mixture to be nebulized. In other words, one handheld nebulizer can satisfy different types of removable cartridge which hold different mixtures. The identification element also minimizes the incidents caused by the user mistakenly inputting the wrong parameters.

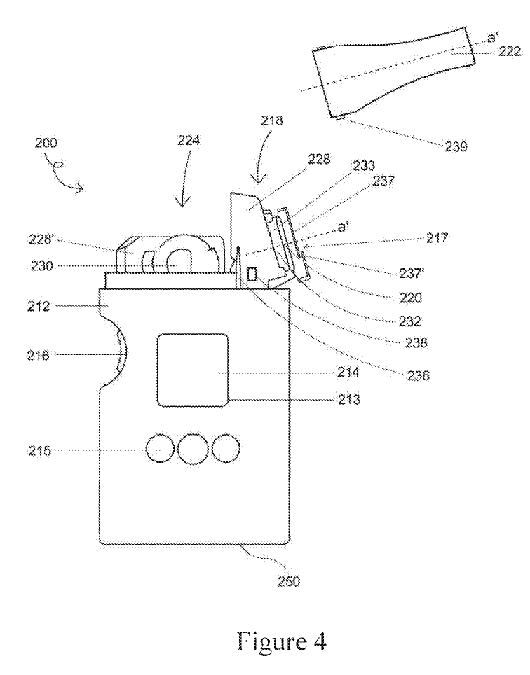

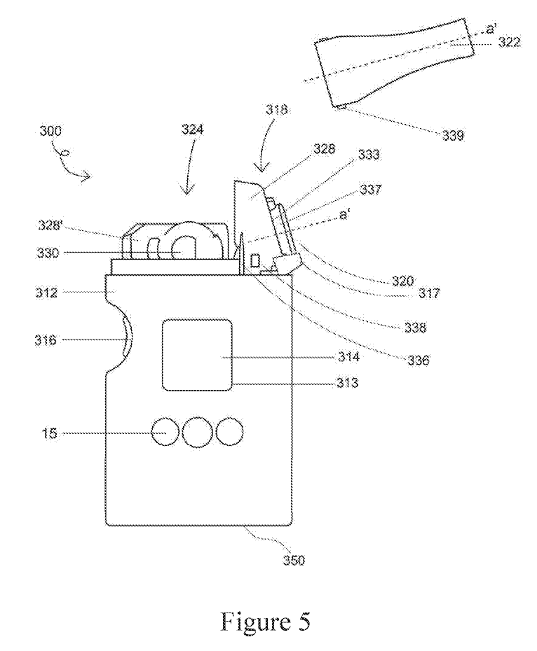

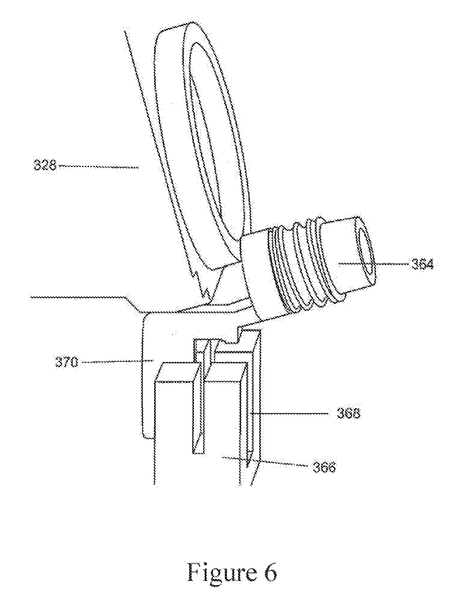

The present invention also provides a handheld nebulizer that can be operated according to a user's breathing, i.e. the handheld nebulizer synchronizes to a user's breathing action, such as their inhalation and exhalation. In the embodiment for FIGS. 4-5, the handheld nebulizer includes an actuation assembly which is responsive to a user's breathing. When the handheld nebulizer is in use, an actuation element of the actuation assembly may detect the air current passing through the guiding nozzle and then sends an actuation signal to the control assembly. The nebulizing element is then actuated to nebulize the mixture in the removable cartridge 228.

FIGS. 4 and 5 show embodiments of a handheld nebulizer having the characteristic of performing nebulization simultaneously with a user's breathing. As shown in FIG. 4, the handheld nebulizer 200 includes most of the components in the handheld nebulizer 100 as shown in FIGS. 1 and 2. The handheld nebulizer 200 includes a body 212 having a control assembly 213 and an actuation assembly which is in electrical communication with the control assembly 213. The actuation assembly 217 is responsive to a user's breathing and includes actuation elements 237, 237′; a nebulizing chamber 218 accommodating a removable cartridge 228 for conducting nebulization, the removable cartridge 228 holds a mixture of a compound to be nebulized, wherein a nebulizing element 232 is affixed or detachably mounted to the removable cartridge 228, and wherein the removable cartridge 228 is in electrical communication with the control assembly 213.

In this embodiment, the actuation elements 237, 237′ are arranged in the nebulizing chamber 218, and positioned between the guiding nozzle 222 and the nebulizing element 232. The actuation elements 237, 237′ are in electrical communication with the control assembly 213. When the user inhales via the guiding nozzle 222, the actuation element 237 shifts to bias against the actuation element 237′ because of the airflow current generated from the user's inhalation. This completes an electrical circuit and an actuation signal is generated and transmitted to the control assembly 213. On the other hand, when the user stops breathing or exhales, the actuation element 237 no longer touches the actuation element 237′, breaking the circuit, and the nebulization is thus stopped. In this embodiment, the guiding nozzle 222 is arranged with a one-way valve 239 on or in the nozzle wall. When the user exhales, the one-way valve 239 opens to discharge the exhaled air so as to reduce or avoid the exhaled air to come into contact with the actuation elements 237, 237′ and/or to enter the nebulizing chamber 218. When the user inhales or stops breathing, the one-way valve 239 is closed. The arrangement of the one-way valve 239 ensures the actuation elements 237, 237′ function normally and react to the user's breathing in a sensitive manner. In one embodiment, the actuation element is between the one way valve and the exit of the guiding nozzle. The one-way valve can be of any design an ordinary skill person in the art can appreciate. For example, the guiding nozzle is arranged with an opening on its wall and a silicone film is arranged to cover the opening.

In an embodiment herein, the actuation element may be a sensor, for example but not limited to an infrared sensor and/or a flow sensor. The sensor may be installed in the nebulizing chamber, or attached to the guiding nozzle to detect the air current passing through the guiding nozzle so as to inform the control assembly and/or the nebulizing element to carry out the corresponding nebulization. When a user's inhalation is detected, a nebulization will be performed simultaneously. When no inhalation is detected, no nebulization will be performed.

As shown in FIG. 5, the handheld nebulizer 300 makes use of a sensor 366 to detect a user's breathing for performing the corresponding nebulization. The handheld nebulizer 300 includes an actuation assembly 317 in the nebulizing chamber 318. The actuation assembly 317 is positioned adjacent to the removable cartridge 328 and includes actuating elements, i.e. an infrared sensor 366 and a movable block 364 (as shown in FIG. 6). With reference to FIG. 6, the movable block 364 can be movably arranged in the recess 368 of the infrared sensor 366. During operation, the infrared sensor 366 generates an infrared light beam and detects, in real time, whether the infrared light beam has passed through the recess. If no infrared light beam is detected, the infrared sensor 366 will send an actuation signal to the control assembly (see 313 in FIG. 5) which is operatively-connected with the infrared sensor 366. When the infrared light beam is detected, the infrared sensor 366 will not send such a signal and/or will stop sending any actuation signal.

When the user inhales, a negative pressure is established in the guiding nozzle 322 and the nebulizing chamber 318. The negative pressure drives the movable block 364 to move towards the user and the tab 370 therefore interrupts the infrared light beam within the infrared sensor 366. The infrared sensor 366 then detects the changes immediately and sends an actuation signal to the control assembly to trigger the corresponding nebulization. The movable block 364 is designed to be particularly sensitive to pressure changes. When the user stops breathing, the movable block 364 returns to its initial position and allows the infrared beam light to pass through the recess 368. The infrared sensor 366 detects the changes (i.e. detects the infrared light) and then stops sending the actuation signal (or sends a stop signal). The nebulization is thus stopped. When the user exhales, provided that a one-way valve 339 is arranged in the guiding nozzle 322, the exhaled air is discharged through the one-way valve 339 without affecting the actuation assembly 317.

In an embodiment herein, the guiding nozzle, 122, 222, 322 may be provided as a disposable form so as to further reduce the chance of contamination.

Based on the disclosure herein, the ordinary person skilled in the art is able to implement the one or more features of the present invention into any known structures of handheld nebulizer for obtaining a handheld nebulizer with substantially improved effects. For example, Chinese Patent No. 103143086B, Chinese Patent No. 103143087B, Chinese Patent No. 103143089B, Chinese Patent No. 103143090B, Chinese Patent No. 103143088B and Chinese Patent No. 103170040B have already provided some possible structures of a handheld nebulizer. The disclosed structures in these publications cannot achieve the desired effects as described in the present invention. Accordingly, the present invention provides possible means and features to improve the existing handheld nebulizer.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

1. A handheld nebulizer comprising:
a body comprising a control assembly and an actuation assembly, wherein the control assembly is in electrical communication with the actuation assembly for delivering nebulized particles of a compound to a subject;
a nebulizing chamber for accommodating a removable cartridge for conducting nebulization, the removable cartridge for holding a mixture of a compound to be nebulized into nebulized particles, wherein a nebulizing element is affixed or detachably mounted to the removable cartridge, and wherein the removable cartridge is in electrical communication with the control assembly.

2. The handheld nebulizer according to claim 1, wherein the nebulizing chamber comprises an outlet, and wherein the outlet is in fluid communication with a guiding nozzle connected to the nebulizing chamber for guiding the flow of the nebulized particles.

3. The handheld nebulizer according to claim 1, wherein the nebulized particles have an average size of about or less than 5 μm.

4. The handheld nebulizer according to claim 1, further comprises a storage chamber for accommodating an additional removable cartridge.

5. The handheld nebulizer according to claim 1, further comprising a heating element for heating the mixture in the removable cartridge before nebulization.

6. The handheld nebulizer according to claim 5, wherein the nebulized particles delivered after the heating and nebulization have a suitable average temperature for the subject to inhale.

7. The handheld nebulizer according to claim 5, wherein the heating element is selected from the group consisting of a heating coil, a heating plate, a copper tube, and a combination thereof.

8. The handheld nebulizer according to claim 6, wherein the average temperature is of from about 25° C. to about 40° C.

9. The handheld nebulizer according to claim 1, further comprising an identification element recognized by the control assembly to determine the identity of the removable cartridge.

10. The handheld nebulizer according to claim 9, wherein the identification element is integrated in to the removable cartridge or is a separate element attached to the removable cartridge.

11. The handheld nebulizer according to claim 9, wherein the identification element is selected from the group consisting of a RFID tag, an electronic chip card, a printed label, a designed structure of the removable cartridge and a combination thereof.

12. The handheld nebulizer according to claim 9, wherein the control assembly adjusts preset parameters of the nebulizer according to the identity of the removable cartridge.

13. The handheld nebulizer according to claim 1, wherein the control assembly comprises a display, a timer, a sensor, an input button, a circuit board, a database or a combination thereof.

14. The handheld nebulizer according to claim 1, wherein the actuation assembly is responsive to a user's breathing, said actuating assembly comprises an actuating element for detecting a change in air current or air pressure.

15. The handheld nebulizer according to claim 14, wherein the nebulizing chamber comprises an outlet in fluid communication with a guiding nozzle connected to the nebulizing chamber, and wherein said guiding nozzle further comprises a one-way valve.

16. A removable cartridge for a handheld nebulizer, comprising a nebulizing element.

17. The removable cartridge according to claim 16, wherein the removable cartridge comprises a mixture of a compound and the nebulizing element nebulizes the mixture to produce nebulized particles with an average size of about or less than 5 μm.

18. The removable cartridge according to claim 16, further comprising a heating element for heating the mixture.

19. The removable cartridge according to claim 16, further comprising an identification element to be recognized by the handheld nebulizer for determining the identity of the removable cartridge.

20. The removable cartridge according to claim 19, wherein the identification element is a designed structure integrated in to the removable cartridge.

21. The removable cartridge according to claim 19, wherein the identification element is selected from the group consisting of a RFID tag, an electronic chip card, a printed label, and a combination thereof.
